(19) United States
(12) Patent Application Publication
(10) Pub. No.: US 2020/0121614 A1
(43) Pub. Date: Apr. 23, 2020

(54) CANNABINOID-INFUSED TRANSPARENT HYDROGEL SKIN PATCH

(71) Applicant: David M. Tumey, Coral Springs, FL (US)

(72) Inventor: David M. Tumey, Coral Springs, FL (US)

(73) Assignee: Bluegrass Farmacueticals, LLC, Belton, KY (US)

(21) Appl. No.: 16/571,717

(22) Filed: Sep. 16, 2019

Related U.S. Application Data
(60) Provisional application No. 62/747,188, filed on Oct. 18, 2018.

(51) Int. Cl. A61K 9/70; A61K 47/69; A61K 9/06; A61K 9/50; A61K 31/05

(52) U.S. Cl. CPC A61K 9/7084; A61K 47/6903; A61K 31/05; A61K 9/50; A61K 9/06

(57) ABSTRACT

A flexible, transparent gel patch product contains a clear cannabinoid-infused hydrogel material in a gel-like phase. The gel patch product is comprised of several layers including a gel layer, removable tab and release liner. The removable tab provides a graspable surface to allow for the removal of the release liner from the gel patch and to facilitate handling of the gel patch during application to the skin. The tab may be comprised of a flat, double-coated paper or other suitable material, or a flexible, V-shaped member, and is removable by peeling after the gel patch is applied. The gel patch includes a transparent cover and a grid pattern may be printed on the backing layer to permit visualization of the underlying skin and measurement of any injury or wounds, for example.

### FIELD OF THE INVENTION

The present general inventive concept relates to hydrogel skin patches and, more particularly, to a transparent, flexible gel patch product containing a cannabinoid infused hydrogel substance.

### BACKGROUND OF THE INVENTION

Exuding dermal injuries, such as cuts, burns, abrasions, decubitus ulcers and open surgical wounds, have traditionally been a medical challenge in providing a sterile barrier for such injuries while maintaining a moist healing environment. The accumulation of wound exudate, such as blood, pustulation and other third-space fluids, in wound pockets promotes growth of bacteria and bio-filmed organisms which cause infection and delay the healing process. However, since it is generally desirable to allow a wound to heal in a slightly moist and occlusive state, as it is understood that this may accelerate healing, excess wound exudate should be removed. If excess wound exudate remains in a wound, a “blister” of exudate can collect under the wound dressing which is not only displeasing, but also may cause the dressing to leak, thereby defeating the objective of sterility. Current methods of aspiration, however, can lead to wound infection or can negatively impact sterility. Further, it is not desirable to remove all of the exudate, as that would result in a dry wound-bed and, hence, create a slower healing process.

Preceding aqueous moisture-absorbing wound dressing methods have additional problems in that the aqueous substrate is generally contained in the center portion of the wound dressing, typically surrounded by a bulky adhesive border, such as a foam border. Problems with such adhesive borders include decreased comfort, conformity and adhesion issues, as well as the potential for a “lifted edge” that could catch on bed sheets or clothing, thereby exposing the wound to bacteria and infection. In addition, observation of the wound by medical caregivers may require removing the wound dressing in part, thereby exposing the wound, again creating a situation where bacteria and infection can be introduced to the wound site.

Adhesive wound dressings, similar to that disclosed by Ward, U.S. Pat. No. 4,753,232, issued Jun. 28, 1988, are frequently constructed of a polymer film having one adhesive surface. The polymer film is typically thin and, therefore, can be difficult to manipulate during application to the wound. Further, it is generally desirable to apply the wound dressing to the patient's skin without touching the part of the dressing surface that is to come into contact with the skin. The prior art discloses several techniques for facilitating handling of the wound dressing. Ward, for example, discloses a “handle” component along one edge of the dressing. After the dressing is applied, the handle may be detached by tearing, or it may include an adhesive coating so that it can be adhered to the skin of the periwound.

An existing method of avoiding contact with the periphery of a bandage by the caregiver's fingers or forceps is disclosed in Brower, U.S. Pat. No. 4,646,731, issued Mar. 3, 1987.

Brower discloses an adhesive-coated bandage whose edges are shielded by a pair of folded V-shaped tabs. After the release liner is removed from the bandage, first one tab is removed and the corresponding end of the bandage is adhered to the skin. The second tab is subsequently grasped and removed as the entire length of the bandage is then applied.

Faasse, Jr., U.S. Pat. No. 4,744,355, issued May 17, 1988, addresses a problem associated with excessive peeling force during removal of release liners from wound dressings. The Faasse patent teaches a release liner adhesively attached to each end of a wound dressing strip (covering layer). A hinge feature is provided between each release liner and the dressing. As the release liners are pulled away from the wound dressing strip, the hinge means are engaged, thus reducing the peeling force on the covering layer and preventing the liners from pulling away from the covering layer prematurely.

United Kingdom Patent Application No. 2,128,479 describes a surgical dressing with two release liner sheets, each covering one half of the dressing and having a free edge radiused at the center of the dressing. As the curved edges of the release liner sheets are peeled back, the center portion of the dressing is applied to the wound, followed by the ends, thereby preserving sterility of the wound by eliminating the need to touch the adhesive surface of the dressing.

European Patent Application Pub. No. 0 168 174 discloses a generally rigid carrier section with bent handles over the outer surface of a thin-film dressing. The carrier section assists in keeping the dressing extended during deployment and prevents the ends from curling in on themselves. The reference further discloses a tab component along one edge of the dressing. This tab component is not coated with adhesive. It may be grasped during application of the dressing to the patient's skin and is detachable afterward by tearing.

U.S. Pat. No. 5,106,629, issued to Cartmell et al. on Apr. 21, 1992, discloses a hydrogel-based wound dressing with a thin-film polymer transparent layer, a dimensionally stable backing layer over the outer surface of the transparent layer, and a release liner. The backing layer and the release liner each have a tab at the corner to permit the peeling away of each from the transparent layer. The hydrogel material is positioned in a central portion of the transparent layer, and the adhesive border portion of the transparent layer adheres to the skin of the periwound. The dimensionally stable backing member prevents the transparent layer from curling and expedites handling of the dressing during its application.

The Cartmell et al. is the only reference to disclose a wound dressing containing a hydrogel material for the absorption of wound exudate. Further, the methods taught by the aforementioned references for facilitating the handling of a thin-film wound dressing layer are generally complicated and may necessitate expense in manufacture and materials, particularly with respect to the overall cost of the wound dressing.

It is clear, therefore, that there exists a need for a hydrogel-based wound dressing product that may be easily handled throughout application to the wound without the need to touch the adhesive side of the dressing. Further, there is a need for a cannabinoid-infused hydrogel gel patch product that is inexpensive and simple to manufacture, and easily removed from a release liner and applied to a wound.

### SUMMARY OF THE INVENTION

The present general inventive concept meets these needs by providing a thin film, transparent gel patch containing a cannabinoid-infused hydrogel material. The present general inventive concept also provides a method of manufacture of the cannabinoid-infused hydrogel product. The gel patch product herein can be manufactured to any desirable size or shape to provide a thin-film, fluid-absorbing gel patch for a wound of any type. The gel patch described herein is transparent, conformable to wear and adheres around its perimeter segment, being nonadhesive directly over the injured skin.

The cannabinoid-infused gel skin patch product of the present general inventive concept comprises a release liner, removable tab, and gel patch. The gel patch comprises a transparent, preferably polyurethane, thin-film layer, a first adhesive layer, a backing layer, second adhesive layer, support layer and a cannabinoid-infused hydrogel material. The transparent film layer, which may be of any desirable shape, but which typically is generally rectangular in shape, has a central portion and a perimeter segment surrounding the central portion, in addition to a first side and an opposing second side. When the gel patch is applied to the skin, the first side of the transparent layer forms the outer surface of the gel patch product. The transparent film layer may, alternatively, be constructed of materials other than polyurethane, such as polyethylene, vinyl, or other appropriate wound dressing materials, and may also be fenestrated in order to allow the patient's skin to breathe.

The first adhesive layer is situated on the second side of the transparent layer. The backing layer, preferably also constructed of transparent polyurethane and having a first side and an opposing second side, is bonded to the second side of the transparent layer by means of the first adhesive layer. A second adhesive layer is positioned on the second side of the backing layer in order to accommodate the first side of the support layer. The support layer is made from a material such as woven and nonwoven fabrics, scrim, cotton gauze or other suitable materials. The cannabinoid-infused hydrogel material is bonded to the second side of the support layer. The porous fabric of the support layer allows the cannabinoid-infused hydrogel material to pass through to the first side of the support layer, resulting in the presence of the hydrogel layer on both sides of the support layer.

The cannabinoid-infused hydrogel material is preferably a normal saline solution in an aqueous gel-like phase, and is contained within the central portion of the transparent polymer film. The gel-like consistency of the hydrogel material creates a releasable gentle bond between the gel patch and the skin without creating an actual adhesive attachment that would damage new tissue growth when removed. An advantage of the gel-like hydrogel is that it will not degrade as the wound fluids are absorbed within. Additionally, it allows clean and orderly removal of the gel patch when the skin has healed or the dressing is changed.

The release liner, which is preferably silicone-coated, overlays the hydrogel material and is bonded to the perimeter segment of the second side of the transparent layer by means of the first adhesive layer. The removable tab is interposed between the transparent layer and the release liner. The tab is adhered to one edge of the perimeter segment of the second side of the transparent layer by means of the first adhesive layer so as to provide a graspable portion to allow for the removal of the release liner from the transparent layer and to facilitate the handling of the gel patch during application of the patch to the skin.

In a preferred embodiment of the present general inventive concept, the backing layer is includes a printed grid pattern to allow medical caregivers to assess the area of damaged skin, without having to remove the gel patch. A clear view of the skin is provided through the gel patch, each layer of which is preferably transparent.

In one embodiment, the removable tab is flat and is constructed of double-coated paper, polystyrene, polyester, or other suitable material. In a second embodiment, the tab comprises a V-shaped member which is preferably silicone-coated. This V-shaped member has a first flap and a second flap, with the first flap being secured to the second side of the transparent layer by means of the first adhesive layer, and the second flap being positioned between the first flap and the release liner. The open end of the V shaped member is arranged along one edge of the transparent layer and the opposing edge of the release liner. In both embodiments, the tab is removable by peeling away after the gel patch is applied to the patient's skin. The tab also assists in adding stability to the thin-film transparent layer as the release liner is removed from the transparent layer.

The present general inventive concept provides a method of manufacturing the cannabinoid-infused hydrogel product. Initially, the transparent polymer film is provided, preferably of a transparent polyurethane material. This film contains central and perimeter segments, along with a first side and an opposing second side. The second side of the transparent film is coated with a preferably medical-grade first adhesive layer. A backing layer is then provided, the second side of which is coated with a preferably medical-grade second adhesive layer. The first side of a support layer is laminated to the second side of the backing layer. The support layer is comprised of material which includes woven and nonwoven fabrics, scrim, cotton gauze or other similar materials, and is permeable to the adhesive.

A cannabinoid-infused hydrogel material is then applied to the second side of the support layer. The interstices within the fabric of the support layer allow the hydrogel material to flow through to the first side of the support layer, such that the hydrogel layer penetrates to both sides of the support layer. Together, the backing layer, support layer and hydrogel material form the reinforced hydrogel patch.

Preferably, the cannabinoid-infused hydrogel patch is manufactured in sheet form and subsequently cut to various sizes and shapes. The size of the transparent layer is determined accordingly in order that the central portion of the transparent layer is capable of accommodating the hydrogel layer. The hydrogel layer is then secured to the transparent layer such that the first side of the backing layer adheres to the central portion of the second side of the transparent layer by means of the first adhesive layer.

A release liner, preferably of a silicone-coated sheet material, is provided. A removable tab having first and second sides is also provided. The first side of the tab is laminated to one edge of the perimeter segment on the second side of the transparent layer, such that the first adhesive layer is situated between the tab and the transparent layer. A first side of the release liner is then laminated to the perimeter segment on the second side of the transparent layer by means of the first adhesive layer. When the gel patch product is completely assembled, the release liner overlies the hydrogel material and patch. Further, the release liner, transparent layer and first adhesive layer form an adhesive seal around the hydrogel material, thereby preserving sterility of the gel patch.

In the preferred method of manufacture, a 1 cm square grid pattern is printed on the backing layer to enable medical caregivers to assess the area of damaged skin through the gel patch. In one embodiment, a flat tab constructed of a double-coated paper is provided. Alternatively, a flat tab constructed of polystyrene, polyester, or other suitable material may be used. In a second embodiment, a tab comprising a V-shaped member is provided and is preferably silicone-coated. The V-shaped member has a first flap and a second flap, and the first flap is bonded to the perimeter segment of the second side of the transparent layer by the first adhesive layer, while the second flap is positioned between the first flap and the release liner with the open end of the “V” positioned along one edge of the transparent layer and the opposing edge of the release liner. In both embodiments, this tab may be removed by peeling away after the gel patch is applied to the patient's skin.

It is an object of the present general inventive concept to provide a gel patch product containing a cannabinoid-infused hydrogel substance which is particularly beneficial when used to dress highly exuding wounds, such as cuts, burns, abrasions or decubitus ulcers, by providing a dermal-like media which is biocompatible, anti inflammatory, fluid-absorbing, and bacterial-protective; to provide a gel patch that is transparent, thereby allowing medical caregivers to observe the healing progression of a wound without removing the dressing; to provide a gel patch that is easily handled and applied to a wound without touching the adhesive portion of the patch; and to provide a gel patch that is inexpensive to manufacture and has greater efficacy than currently marketed hydrogel patches.

Other objects and advantages of the present general inventive concept will be apparent from the following description, the accompanying drawings and the appended claims.

### BRIEF DESCRIPTION OF THE DRAWINGS

FIG. 1 is a perspective view of the cannabinoid-infused hydrogel product.

FIG. 2 is an exploded perspective view, illustrating the layers which form a preferred embodiment of the cannabinoid-infused hydrogel product.

FIG. 3 is an exploded side view of the cannabinoid-infused hydrogel product of FIG. 2.

FIG. 4 is a cross-sectional view of the cannabinoid-infused hydrogel product of FIG. 1 taken along line 4-4.

FIG. 5 is a side view of the cannabinoid-infused hydrogel product which illustrates the peeling of the release liner from the gel patch.

FIG. 6 is a perspective view showing the cannabinoid-infused hydrogel patch in place on the patient's skin.

FIG. 7 illustrates a second preferred embodiment in which the flat, polystyrene tab of FIGS. 1-6 is replaced with a V-shaped tab.

FIG. 8 illustrates a sectional view aspect the second preferred embodiment in FIG. 7.

FIG. 9A illustrates a first step preferred method of application of the cannabinoid infused hydrogel product of the present general inventive concept.

FIG. 9B illustrates a second step preferred method of application of the cannabinoid infused hydrogel product of the present general inventive concept.

FIG. 9C illustrates a third step preferred method of application of the cannabinoid infused hydrogel product of the present general inventive concept.

FIG. 9D illustrates a fourth step preferred method of application of the cannabinoid infused hydrogel product of the present general inventive concept.

### DETAILED DESCRIPTION OF THE PREFERRED EMBODIMENT

The present general inventive concept relates to a cannabinoid-infused hydrogel product for application to skin which is comprised of a thin-film transparent layer and a cannabinoid-infused hydrogel layer. The invention also includes a method of manufacture for the disclosed cannabinoid-infused hydrogel product.

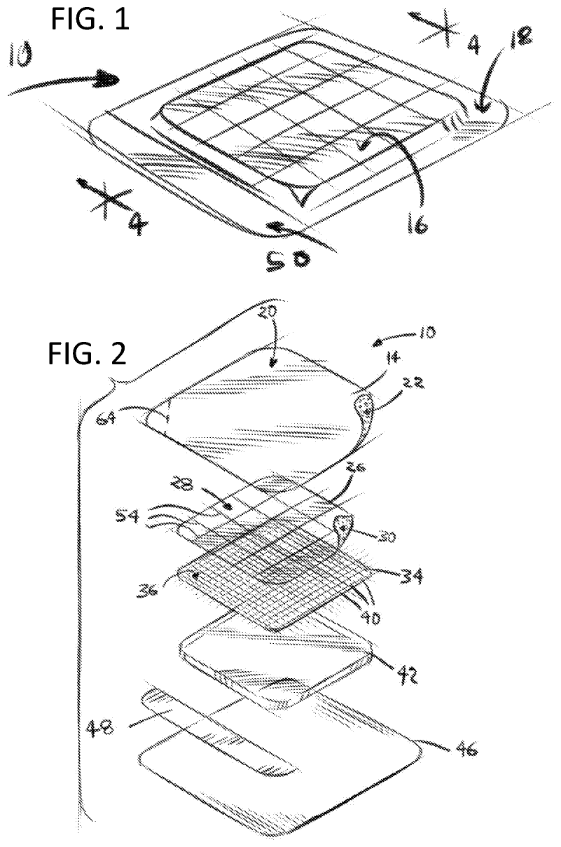

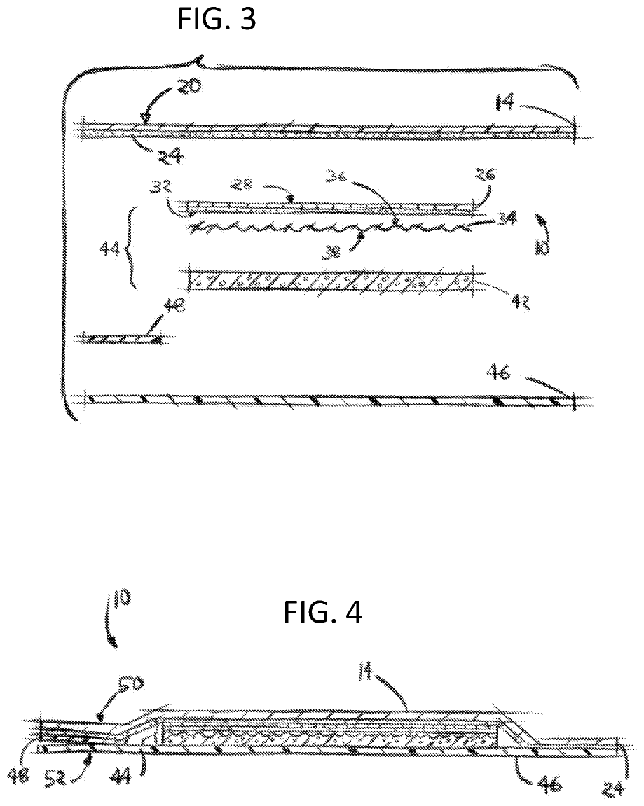

Referring to FIG. 1, the cannabinoid-infused hydrogel product 10 of the present general inventive concept is depicted. Although the cannabinoid-infused hydrogel product 10 illustrated in FIG. 1 portrays a rectangular shape, it may be any of a variety of desired shapes. The cannabinoid-infused hydrogel product 10 is composed of several layers, as illustrated by the exploded view of FIG. 2 and the exploded side view of FIG. 3.

Referring, collectively, to FIGS. 1, 2 and 3, the cannabinoid-infused hydrogel product 10 includes a thin-film transparent layer 14, preferably of polyurethane, which has a central portion 16 and a perimeter segment 18. The transparent layer 14 has a first side 20 and a second side 22, the second side 22 being coated with a first adhesive layer 24. Backing layer 26, preferably composed of transparent polyurethane and having a first side 28 and a second side 30, is adhered to the second side 22 of transparent layer 14 by means of first adhesive layer 24. A second adhesive layer 32 is situated on the second side 30 of backing layer 26 in order to accommodate first side 36 of a support layer 34. The support layer 34 is made from a material such as woven and nonwoven fabrics, scrim, cotton gauze or other similar materials.

A cannabinoid-infused hydrogel material 42 is bonded to the second side 38 of support layer 34. The permeable fabric of the support layer 34 contains interstices 40 which allow the hydrogel layer 42 to pass through to the first side 36 of support layer 34, resulting in the presence of hydrogel layer 42 on both the second side 38 and the first side 36 of support layer 34. The cannabinoid-infused hydrogel material 42 is preferably a normal saline solution in an aqueous gel-like phase. The hydrogel material 42, support layer 34 and backing layer 26 together form a reinforced hydrogel scaffold 44, which is contained within the central portion 16 of transparent layer 14. A release liner 46, preferably of a silicone-coated sheet material, overlies the hydrogel material 42 and is secured to the perimeter segment 18 of the second side 22 of transparent layer 14 by means of first adhesive layer 24.

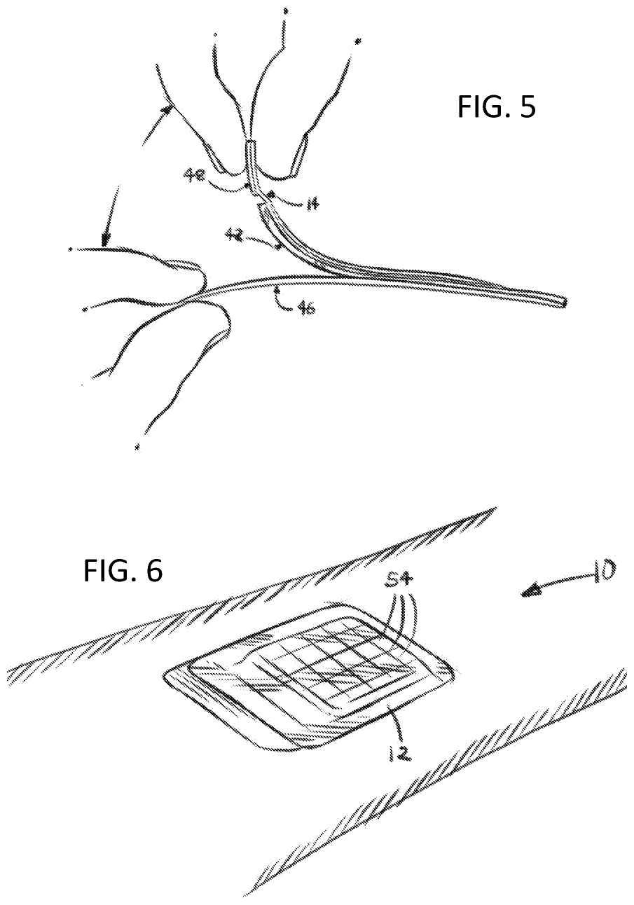

Referring now to FIGS. 4-6, collectively, a removable tab 48 is interposed between the transparent layer 14 and the release liner 46. The tab 48 is adhered to one edge 50 of the perimeter segment 18 of transparent layer 14 by means of the first adhesive layer 24, so as to provide a graspable surface to allow for the removal of the release liner 46 from transparent layer 14 and to promote the handling of the gel patch 12 during application of the gel patch 12 to the patient's skin.

The gel-like consistency of the cannabinoid-infused hydrogel material 42 creates a releasable bond between the gel patch 12 and the skin without creating an actual adhesive attachment that would damage new tissue growth upon removal. An advantage of the gel-like hydrogel material 42 is that it will not degrade as the wound fluids are absorbed. Additionally, it permits clean and orderly removal of the gel patch 12 when the wound heals or the patch 12 is changed.

In the preferred embodiment of the present general inventive concept, the backing layer 26 is printed with a square grid pattern 54 to allow medical caregivers to assess the area of damaged skin, without having to remove the gel patch 12. A clear view of the skin is provided through the patch 12, each layer of which is preferably transparent. Although FIGS. 1, 2, 6 and 7 portray a rectangular grid pattern 54, any suitable grid pattern may be incorporated.

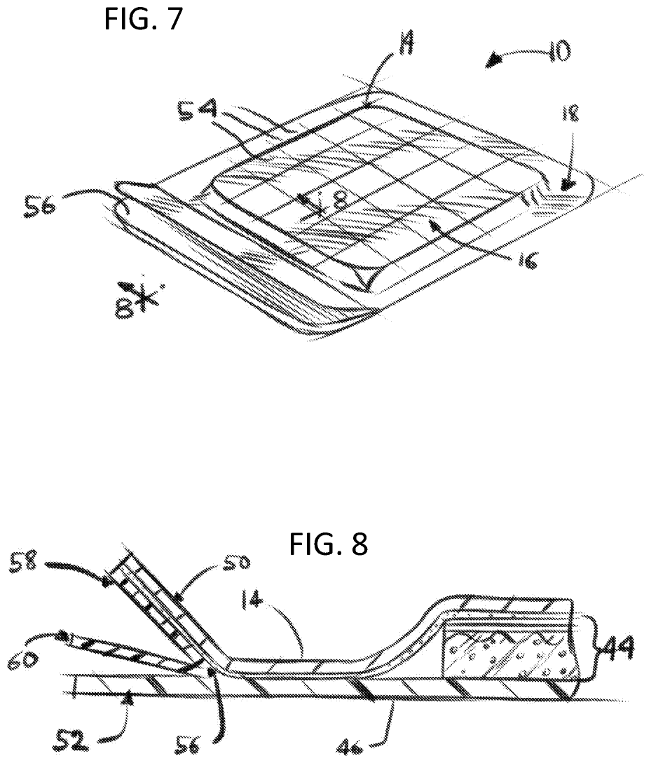

In one embodiment of the present general inventive concept, the removable tab 48 is constructed of a double-coated paper, polystyrene, polyester, or other suitable material, and is preferably flat, as shown in FIGS. 1-6. FIGS. 7 and 8 illustrate a second embodiment, wherein the flat tab 48 is replaced with a V-shaped member 56. The V shaped member 56 has a first flap 58 and a second flap 60, with the first flap 58 being bonded to one edge 50 of the second side 22 of transparent layer 14 by means of first adhesive layer 24, and the second flap 60 being disposed between first flap 58 and one edge 52 of release liner 46. The V-shaped member 56 and the release liner 46 are preferably coated with silicone. This enables the V-shaped member 56 to be more readily removed from the transparent layer 14, and enables the release liner 46 to be more readily removed from the transparent layer 14 and cannabinoid-infused hydrogel material 42. Both the tab 48 and the V-shaped member 56 are removable by peeling away after the gel patch 12 is applied to the wound site. The tab 48 and V-shaped member 56 also assist in adding stability to the transparent layer 14 as release liner 46 is removed from the cannabinoid-infused hydrogel product 10.

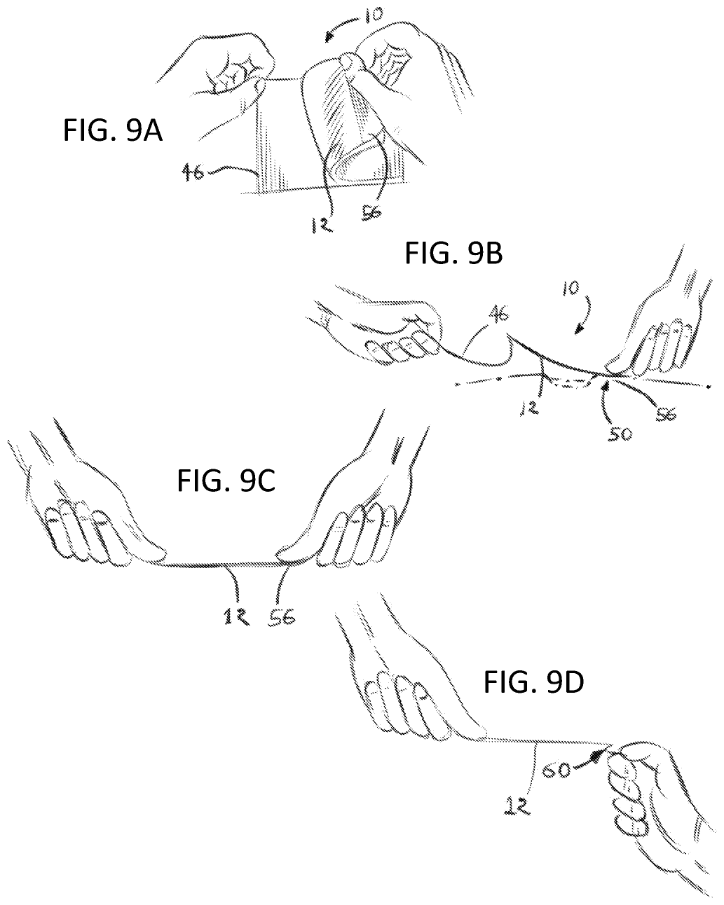

FIGS. 9A through 9D illustrate the preferred method of application of the cannabinoid-infused hydrogel product 10 to the skin. Although these figures illustrate the application of a gel patch 12 having a V-shaped member 56, a similar procedure may be used to apply the gel patch 12 of FIGS. 2-5 wherein a flat tab 48 is employed. As shown in FIG. 9A, the release liner 46 is first removed from the gel patch 12 by grasping V-shaped member 56 and one edge 50 of transparent layer 14 with one hand, while grasping release liner 46 with the other hand. After the edge 50 of transparent layer 14 is removed from release liner 46, edge 50 is applied to the periwound skin surrounding the wound of the patient. Edge 50 is then held in place while the release liner 46 is fully removed from gel patch 12, as shown in FIG. 9B. After release liner 46 is removed, the gel patch 12 is sealed to the skin, as illustrated in FIG. 9C. As shown in FIG. 9D, V-shaped member 56 is then removed by pulling flap 60 with one hand while the opposite end of transparent film 14 of gel patch 12 is held against the skin by the other hand.

The present general inventive concept also provides a method of manufacturing the cannabinoid-infused hydrogel product 10. Initially, a transparent thin-film polymer layer 14 is provided, preferably of a polyurethane material. Alternatively, transparent layer 14 may be constructed of polyethylene, vinyl, or any other suitable material, and may be fenestrated throughout to allow air to contact the patient's skin. This transparent layer 14 contains a central portion 16 and a perimeter segment 18, along with a first side 20 and a second side 22. The second side 22 of the transparent layer 14 is coated with a preferably medical-grade first adhesive layer 24. A backing layer 26 is then provided, the second side 30 of which is coated with a preferably medical-grade second adhesive layer 32. The first side 36 of a support layer 34 is laminated to the second side 30 of the backing layer 26. The support layer 34 comprises a material such as woven and nonwoven fabrics, scrim, cotton gauze or other similar materials. A cannabinoid-infused hydrogel material 42 is then applied to the second side 38 of the support layer 34. The support layer 34 is permeable and contains interstices 40 within the fabric which allow the hydrogel layer 42 which, when applied to support layer 34, is in liquid form to flow through to the first side 36 of the support layer 34, such that the hydrogel layer 42 is present on both the first side 36 and the second side 38 of the support layer 34. Together, the backing layer 26, support layer 34 and hydrogel material 42 form a reinforced hydrogel scaffold 44.

The hydrogel scaffold 44 is preferably assembled in a sheet form and subsequently cut into various pieces of desired size and shape. The size and shape of the transparent layer 14 are selected so as to accommodate the hydrogel scaffold 44. The hydrogel scaffold 44 is then laminated to the central portion 16 of transparent layer 14 such that the first side 28 of backing layer 26 adheres to the second side 22 of transparent layer 14 by means of first adhesive layer 24.

A release liner 46, preferably coated with silicone, is provided. A removable tab 48 having first and second sides is also provided. The first side of tab 48 is laminated to one edge 50 of the perimeter segment 18 on the second side 22 of transparent layer 14, such that the first adhesive layer 24 is disposed between tab 48 and transparent layer 14. A first side of release liner 46 is then laminated to the perimeter segment 18 on the second side 22 of transparent layer 14 by means of first adhesive layer 24. When the cannabinoid-infused hydrogel product 10 is completely assembled, the release liner 46 overlies the hydrogel material 42 and hydrogel scaffold 44. In fact, the release liner 46, transparent layer 14 and first adhesive layer 24 form an adhesive seal around hydrogel material 42, thus preserving the sterility of the cannabinoid-infused hydrogel product 10.

The preferred cannabinoid-infused hydrogel material 42 is formed from an aqueous mixture including from about 0% to about 90% by weight polyhydric alcohol; from about 6% to about 60% by weight aliphatic diisocyanate-terminated prepolymer; from about 4% to about 40% by weight polyethylene oxide-based polyamine; up to about 2% by weight sodium chloride; and the balance distilled water into which a tincture containing between 10 mg and 30 mg of cannabidiol isolate is mixed. A more preferred hydrogel composition for forming hydrogel layer 42 comprises from about 15% to about 30% by weight of a polyhydric alcohol selected from a group consisting of polypropylene glycol, polyethylene glycol and glycerine, from about 8% to about 14% by weight isophoronediisocyanate-terminated prepolymer, from about 5% to about 10% by weight polyethylene oxide-based diamine, up to about 1% by weight of a salt, and the remaining percentage distilled water into which a tincture containing between 15 mg and 30 mg of cannabidiol isolate is mixed. Alternatively, the cannabidiol isolate may be microencapsulated with a water soluble aqueous coating material, using myriad techniques well known to anyone of ordinary skill in the art. Additionally, the cannabidiol may be mixed throughout the hydrogel or concentrated at the skin-facing interface, the latter configuration being achieved by applying the cannabidiol to the skin-facing surface of the hydrogel prior to, during or after curing. Most preferably, the hydrogel material 42 includes 17% propylene glycol, 12% isophoronediisocyanate-terminated prepolymer, 9% polyethylene oxide-based diamine, 1% salt, and 61% distilled water into which a tincture containing 20 mg of cannabidiol isolate is mixed. The hydrogel material 42 provides biocompatible, nonirritating, fluid-absorbing, bacterial-protective, cushioning, dermal-like media with powerful anti-inflammatory effect due to the cannabidiol.

In the preferred method of manufacture, a square grid pattern 54 is printed on backing layer 26 to enable medical caregivers to assess the underlying skin. Those skilled in the art will recognize that a similar grid pattern could be printed on the transparent layer 14, rather than on the backing layer 26, without deviating from the general inventive concept. In one embodiment, a flat tab 48, constructed of a double coated paper, polystyrene, polyester, or any other suitable material, is provided. This first embodiment may further include a transparent layer 14 having a cropped corner 64, as represented by the phantom line drawn in FIG. 2, at the edge 50 of the perimeter segment 18, in order to facilitate removal of the tab 48 from transparent layer 14 after gel patch 12 is applied to the skin. In a second embodiment, a V-shaped member 56, preferably coated with silicone, is provided in place of flat tab 48. The V-shaped member 56 has a first flap 58 and a second flap 60. The first flap 58 is secured to the second side 22 of the transparent layer 14 by first adhesive layer 24, while the second flap 60 is disposed between the first flap 58 and release liner 46. The open end of the “V” is positioned along one edge 50 of transparent layer 14 and the opposing edge 52 of release liner 46. Both the tab 48 and the V-shaped member 56 are removable by peeling away after the gel patch 12 is applied to the patient's skin.

The cannabinoid-infused hydrogel product 10 of the present general inventive concept is particularly beneficial for use on highly exuding wounds. In particular, a special feature of the hydrogel material 42 is that it is sufficiently clear and transparent that visual observation of the underlying skin is possible without removal of the gel patch 12. Another benefit of the hydrogel material 42 is that it retains its gel-like integrity even upon removal of the gel patch 12 from a wound site. The hydrogel material 42 does not leave any debris in the wound when the gel patch 12 is removed, nor does it adhere to the wound site. The benefit of this feature is that the hydrogel material 42 exhibits a capability of non-traumatically releasing from the skin when the gel patch 12 is removed, so as to not destroy new tissue growth forming at the wound site. Thus, healing is not inhibited by removal of the gel patch 12.

The above described embodiments are set forth by way of example and are not for the purpose of limiting the scope of the present general inventive concept. It will be readily apparent that obvious modifications, derivations and variations can be made to the embodiments without departing from the scope of the invention. Accordingly, the claims appended hereto should be read in their full scope including any such modifications, derivations and variations.

What is claimed is:

1. A cannabinoid-infused hydrogel skin patch, which includes:
a thin-film cover layer having a first side coated with a first adhesive layer;
a backing layer having a first side connected to said first adhesive layer and having a second side coated with a second adhesive layer;
a support layer having a first side and and a second side, said first side connected to said second adhesive layer wherein said support layer is made of permeable fabric having interstices; and
a cannabinoid-infused hydrogel material bonded to said second side of support layer in a manner such that said cannabinoid-infused hydrogel material permeating on said second side and said first side of support layer.

2. The cannabinoid-infused hydrogel skin patch of claim 1, wherein said support layer is made from a material such as woven and nonwoven fabrics, scrim, cotton gauze or other similar materials.

3. The cannabinoid-infused hydrogel skin patch of claim 1, wherein said thin-film cover layer and said backing layer are made of a transparent material.

4. The cannabinoid-infused hydrogel skin patch of claim 3, wherein said transparent material includes one of polyurethane, vinyl, and polypropylene.

5. The cannabinoid-infused hydrogel skin patch of claim 1, wherein said thin-film cover layer and said backing layer include a rectangular grid pattern.

6. The cannabinoid-infused hydrogel skin patch of claim 1, wherein said cannabinoid-infused hydrogel material is bonded to said support layer and said interstices allow said hydrogel layer to pass therethrough wherein said hydrogel material, said support layer and said backing layer together form a reinforced hydrogel scaffold.

7. The cannabinoid-infused hydrogel skin patch of claim 3, wherein said cannabinoid-infused hydrogel material is bonded to said support layer and said interstices allow said hydrogel layer to pass therethrough wherein said hydrogel material, said support layer and said backing layer together form a reinforced hydrogel scaffold.

8. The cannabinoid-infused hydrogel skin patch of claim 7, wherein said hydrogel material, said support layer and said backing layer together form a reinforced hydrogel scaffold, which is contained within a central portion of said transparent layers.

9. The cannabinoid-infused hydrogel skin patch of claim 1, wherein said cannabinoid-infused hydrogel material includes a saline solution in an aqueous gel-like phase.

10. The cannabinoid-infused hydrogel skin patch of claim 1, which further includes a release liner overlying said cannabinoid-infused hydrogel material.

11. The cannabinoid-infused hydrogel skin patch of claim 1, wherein said thin-film cover layer has a perimeter portion extending beyond said backing layer, said support layer and said cannabinoid-infused hydrogel material providing for connection to skin surrounding a wound.

12. The cannabinoid-infused hydrogel skin patch of claim 11, wherein said thin-film cover layer has a perimeter portion extending beyond said backing layer, said support layer and said cannabinoid-infused hydrogel material providing for connection to skin surrounding a wound and wherein release liner overlies said perimeter portion.

13. The cannabinoid-infused hydrogel skin patch of claim 10, wherein said release liner includes a silicone-coated sheet material.

14. The cannabinoid-infused hydrogel skin patch of claim 12, which includes a removable member disposed between said perimeter portion and said release liner to form a tab.

15. The cannabinoid-infused hydrogel skin patch of claim 14, wherein said removable member is constructed of one of a double coated paper, polystyrene, polyester.

16. The cannabinoid-infused hydrogel skin patch of claim 1, wherein said cannabinoid-infused hydrogel material is formed from an aqueous mixture including from about 0% to about 90% by weight polyhydric alcohol; from about 6% to about 60% by weight aliphatic diisocyanate-terminated prepolymer; from about 4% to about 40% by weight polyethylene oxide-based polyamine; up to about 2% by weight sodium chloride; and a balance of distilled water into which a tincture containing between 10 mg and 30 mg of cannabidiol isolate is admixed.

17. The cannabinoid-infused hydrogel skin patch of claim 1, wherein said cannabinoid-infused hydrogel material is formed from about 15% to about 30% by weight of a polyhydric alcohol selected from a group consisting of polypropylene glycol, polyethylene glycol and glycerine, from about 8% to about 14% by weight isophoronediisocyanate-terminated prepolymer, from about 5% to about 10% by weight polyethylene oxide-based diamine, up to about 1% by weight of a salt, and the remaining percentage distilled water into which a tincture containing between 15 mg and 30 mg of cannabidiol isolate is admixed.

18. The cannabinoid-infused hydrogel skin patch of claim 1, wherein said cannabinoid-infused hydrogel material is formed 17% propylene glycol, 12% isophoronediisocyanate-terminated prepolymer, 9% polyethylene oxide-based diamine, 1% salt, and 61% distilled water into which a tincture containing 20 mg of cannabidiol isolate is admixed.

19. The cannabinoid-infused hydrogel skin patch of claim 1, wherein said cannabinoid-infused hydrogel material is formed from about 15% to about 30% by weight of a polyhydric alcohol selected from a group consisting of polypropylene glycol, polyethylene glycol and glycerine, from about 8% to about 14% by weight isophoronediisocyanate-terminated prepolymer, from about 5% to about 10% by weight polyethylene oxide-based diamine, up to about 1% by weight of a salt, and the remaining percentage distilled water into which water soluble microencapsulated cannabidiol isolate is admixed.

20. The cannabinoid-infused hydrogel skin patch of claim 19, wherein the water soluble microencapsulated cannabidiol isolate is concentrated primarily at the skin-facing surface of the skin patch.
